(19) United States
(12) Patent Application Publication
(10) Pub. No.: US 2019/0382699 A1
(43) Pub. Date: Dec. 19, 2019

(54) MICROORGANISM TEST SYSTEM

(71) Applicant: SENSIRION AG, Stäfa (CH)

(72) Inventor: Matthias STREIFF, Stäfa (CH)

(73) Assignee: SENSIRION AG, Stäfa (CH)

(21) Appl. No.: 16/464,084

(22) PCT Filed: Nov. 24, 2016
(86) PCT No.: PCT/EP2016/078721

§ 371 Date: May 24, 2019

(51) Int. Cl. C12M 1/34; C12Q 1/06; C12M 1/00; C12Q 1/24; B01L 3/00

(52) U.S. Cl. CPC C12M 1/34; C12Q 1/06; C12M 47/04; B01L 2300/123; B01L 3/50255; B01L 2300/0681; C12Q 1/24

(57) ABSTRACT

A device for processing microorganisms comprises a channel comprising an inlet for introducing a fluid sample into the channel, and an outlet. The channel is dimensioned to hold, between the inlet and the outlet, a volume in a range between 1 nl and 50 μl of fluid. A size selective filter is arranged at the outlet for retaining microorganisms (M) in the channel. The size selective filter comprises pores of a size smaller than an average size of the microorganisms (M) to be processed.

### TECHNICAL FIELD

The invention relates to a device, an apparatus and a method for processing microorganisms.

### BACKGROUND ART

In the medical field it is common practice to investigate a body fluid sample taken from a human being or an animal suffering from a disease. It is desired to detect pathogenic organisms in the body fluid sample possibly causing the disease in order to suggest antibiotics or other reagents to treat the disease.

Instruments supporting such investigations are known in the art and are commercially available, such as the Becton Dickinson™ Phoenix™ or the Biomerieux™ Vitek™ 2.

However, such instruments require cultures of microorganisms as inputs that have to be grown beforehand in different other instruments like BactALERT™, or BACTEC™ systems to around 0.5 McFarland. This typically takes one or two days in advance in a good case and a week or more in unfavorable cases. The shorter the time to identify a curing antibiotic, the better for a patient with a bacterial or fungal infection. In most cases shortening that time even by hours matters. In addition, such instruments are bulky, amongst others not suitable for point of care testing.

### SUMMARY OF THE INVENTION

Therefore, it is desired to provide a device and a method enabling a processing of a fluid sample, in particular a body fluid sample, within short processing times. In particular, it is also desired to concentrate fluid samples comprising a low microbial load to a higher concentration of microorganisms per volume unit. Subsequent steps to such concentration may include finding an antimicrobial treatment against the microorganisms present in the fluid sample and/or identification of the type of microorganisms present in the fluid sample.

Such antimicrobial treatment or identification of microorganisms may be desired for treatment of a patient suffering from a microbial infection or for other applications such as e.g. for the treatment of drinking water or another water body or a food. Accordingly, in some embodiments the fluid sample processed may be obtained e.g. from a swimming pool, a water treatment system, another water body, a liquid food or any liquid extract or eluent of a material or substance potentially contaminated with microorganisms. This problem is solved by the device of claim 1. Accordingly, the device for processing microorganisms comprises a channel comprising an inlet for introducing a fluid sample into the channel, and an outlet.

The fluid sample preferably is a liquid sample, and preferably is a sample of a body fluid such as urine, spinal fluid, or, where applicable blood serum or blood pretreated to avoid blood clotting. If the suspected microorganisms are separable from blood cells, a blood sample may also be pretreated e.g. by a bead based affinity “clean up step”, or by an immunofiltration method in order to remove the blood cells.

Microorganisms present in such otherwise sterile fluid sample may be bacteria, fungi, protozoa, algae, or archaea, however, for medical applications most likely bacteria or fungi or protozoa. Microorganisms targeted by the device, apparatus and method according to the invention include unicellular and multicellular organisms and sometimes in this text are referred to simply as cells. Generally bacterial infections or contaminations can be based on a wide range of bacteria, including common bacteria such as E. Coli, Salmonella, Staphylococcus and others. However, a microorganism-bead cluster shall also be represented by the term microorganism. In order to generate a microorganism-bead combination, a bead such as an affinity-molecule coated micro-bead, e.g. a micro-bead, coated with an antibody, with a diameter in the range of 1 μm to 10 μm or even less than 1 μm, then being referred to as nano-bead, may be brought into the fluid sample and results in the attaching of a micro-organism to the bead. This is the procedure referred to above as clean-up.

Prior art systems processing or analyzing fluids with a low microbial load require growing the number of microorganisms by in vitro culture. In particular, if a concentration of microorganisms in a fluid sample taken from the body is too small, for prior art devices lengthy incubation times for in vitro increasing the number of microorganisms in the sample are required for generating a concentration of microorganisms sufficient to be investigated. Instead, in the present device the microorganisms are concentrated within a small volume, i.e. the channel, by injecting a fluid sample into the channel and filtering the individual microorganisms to remain in the channel thereby reaching a desired concentration of microorganisms. The concentration can be as small as a single microorganism per volume unit, in particular volume of the channel, however, preferably more than one, and in a preferred embodiment between 10 and 100 microorganisms. In a preferred embodiment, the channel dimensions and geometry may be adapted to the expected number of microorganisms in the channel, and, for example, may correspondingly may be of a volume to allow for an average acceptance of 10 or hundred microorganisms. Specifically, the channel may be dimensioned in width and height to only allow a single microorganism per longitudinal unit such that the microorganisms are aligned in sequence in the channel.

The volume of the channel preferably is dimensioned to hold, between the inlet and the outlet, a volume in the range of 1 pl and 100 μl of fluid, more preferably in the range of 1 nl and 50 μl, more preferably in the range of 1 nl and 100 nl, and most preferably in the range of 1 and 3 nl. Hence, a small number of microorganisms, e.g. 1 to 100 microorganisms, in the original fluid sample is concentrated into a very small volume, e.g. in the order of order 1 nl. Such concentration is considered as sufficient to obtain a measure of the presence of the microorganisms, in particular obtain a quantitative measure of the number of microorganisms or the metabolic activity of the microorganisms present e.g. by a chemical reaction of a reagent with the microorganisms such as a dye and/or to detect directly or indirectly a metabolic reaction product of the microorganisms e.g. in the presence or absence of an antimicrobial drug such as an antibiotic.

For retaining the microorganisms in the channel, a size selective filter is arranged at the outlet of the channel. The size selective filter preferably is made from a material that is inert, i.e. a material that is not or is not substantially reactive. Hence, the size selective filter performs a mechanical separation between particles allowed to pass the filter and others prevented from passing the filter. Hence, the selection criterion is the size of the particle. The particles to be prevented from passing the filter are the microorganisms. Hence, pores, or openings in the filter are dimensioned to retain at least a majority of the microorganisms desired to be collected in the channel. In case, specific microorganisms are expected to be detected in the fluid sample and to be collected in the channel, the pore size of the filter may be dimensioned with respect to the size of this specific microorganism, for example. However, in a more general approach, an estimated size of the smallest microorganisms to be detected is determined upfront and the pores of the filter are dimensioned smaller, and preferably, 20% smaller, with respect to this size. The size of a microorganism may be understood as a diameter, and preferably as a smallest diameter of the microorganism for the determination of the pore size. In a preferred embodiment, the size selective filter comprises pores with a diameter in the range between 0.01 μm and 10 μm, preferably between 0.1 μm and 1 μm. This dimensioning of pores proves to retain most of the different bacteria expected in a body fluid sample.

The fluid sample as such, i.e. without micro-organisms, or at least portions of it, may leave the channel via the outlet through the filter during this phase of collecting microorganisms in the channel. In one embodiment of the present invention, the filter is arranged in parallel with respect to the flow direction of the fluid, such that the device represents a cross flow filter. Generally, the entire device can be considered as a filter device for filtering microorganisms out of a fluid sample.

In the specific case of the microorganisms being microorganism-bead combinations, and even more specifically when the associate bead has a magnetic property, the microorganisms may also be retained in the channel by means of a magnet provided in the device or the apparatus. In this scenario, the filter at the outlet may be redundant, i.e. it may be abstained from the filter at the outlet. However, in a different embodiment, the filter still is provided at the outlet in addition to the magnet, which may be a permanent magnet or an electromagnet.

The channel has an inlet for accepting the fluid sample and an outlet. The inlet may in one embodiment show an interface for attaching an injector for injecting the fluid sample into the channel. For example, the injector may be a syringe or a pump containing the fluid sample. However, in a different embodiment, and as will be explained later on, in case of a multi-device apparatus, the interface may rather be provided once by the apparatus for providing the fluid sample to the various devices simultaneously such that in this case the individual device not necessarily requires such interface.

In a preferred embodiment, a detector is provided for detecting microorganisms in the fluid sample in the channel. This detector may in one embodiment only detect the presence of absence of microorganisms, and possibly an increase or decrease of microbial biomass. In a different embodiment, the detector may also quantify the number of microorganisms. In such event, the detector is also referred to as counter. Still it comprises such sensor.

In the specific case of the microorganisms being microorganism-bead combinations, and even more specifically when the associate bead has a magnetic property, the sensor may be a sensor for detecting such magnetic property, e.g. a magneto-resistive sensor or a magnetic field sensor. This sensor rather detects the magnetic beads than the microorganisms. This sensor may allow to detect the presence or absence of beads and, hence, microorganisms in the channel, or may even allow the counting of beads, and, hence, microorganisms in the channel. For the latter, it can be assumed that, there is an certain average number of microorganisms linked to a bead within an experiment such that the bead count per channel can be used as a representation of the count of microorganisms in that channel with a certain error. In order to obtain an even more accurate count a secondary optical label attached to or introduced in the microorganism in question can be quantified by optical excitation and will give a signal proportional to the number of microorganisms present. Such a label can be an antibody affine to a membrane protein of the microorganism labelled with a fluorophore or a fluorescent dye that is able to stain the microorganism itself.

Preferably, an electrically controllable valve is provided for closing the inlet in response to a signal supplied by the detector, hence, in one embodiment, if microorganisms are detected, the valve may be activated, and preferably closed. In case of the detector being a counter, the counter may provide a counting result, and in case the counting results exceeds a threshold, the valve may be activated, and preferably closed. In case of a counter, the counting may include counting the number or microorganisms when entering the volume. In this embodiment, a sensor for identifying a microorganism may be arranged close to the inlet in order to detect every microorganism in the fluid sample entering the channel. In a different embodiment, the sensor may be arranged at a different position along the channel, thereby counting the microorganisms when passing this particular position. In a different embodiment, the sensor of the counter may cover the entire channel at once and allow to determine the overall number of microorganisms present in the channel in a single measurement. Hence, the detection of the microorganisms in the channel may not only include the counting of individual microorganisms passing a defined location of the channel but also a collective counting in a single measurement step. Again, in case the sensor cannot perform the individual counting of microorganisms, such sensor, even when covering the entire channel, may provide the information as to absence or presence of microorganisms in the channel, and possibly an increase or decrease of microbial biomass in the channel. All these measurements aiming at counting the microorganisms allow for determining the number of microorganisms present in the fluid sample introduced into and retained within the channel. Thus, the original fluid sample may leave the channel through the outlet, while the number of microorganisms that populated the original fluid sample passing through the channel are collected in the channel and remain there.

In a preferred embodiment, the detector includes a sensor for measuring a dielectric such as an impedance sensor or a capacitive sensor. In impedance measuring, the microorganism representing a dielectric particle becomes polarized in the presence of an electric field. The polarization which is the dipole moment per unit volume depends on the electric field applied and an effective permittivity of the system and the electric field.

In measuring impedance, which is also referred to as impedance spectroscopy, an alternating electrical field (kHz, MHz) is applied to the fluid to be investigated. From Maxwell-Garnett mixing equation it can be deduced that microorganisms contribute an additive dipole moment. The change in the permittivity arising from the additional dipole moment of the microorganism is investigated. The included dipole superimposes to the applied electrical field and hence can be detected.

For determining the impedance, electrodes of the sensor are preferably arranged such that a portion of the channel is exposed to an alternating electrical field.

In a first embodiment, the electrodes are arranged vertical with respect to each other, hence a first electrode is arranged on top of the channel and a second electrode is arranged at the bottom of the channel, e.g. on a substrate.

In a different embodiment, the electrodes are arranged in the same plane, e.g. at the bottom of the channel, which facilitates manufacturing. An embodiment of such electrode arrangement is also referred to as liquid electrodes. Here, the metal electrode coverage is removed from the immediate vicinity of the channel, while the electrical field is conducted to the channel by narrow guides, such that the strength of the electrical field the fluid in the channel is exposed to is sufficient.

Even if not arranged as liquid electrodes, it is preferred that the electrodes are arranged in a common plane. In one embodiment, the electrodes are arranged at both sides of the channel, i.e. lateral to the channels longitudinal extension. In this embodiment, the fluid takes the microorganisms through the measurement area, where an electrical field is applied across the channel, i.e. transverse to the channels longitudinal extension. When an alternating electrical field is applied to the electrodes and a particle such as a microorganism enters the electrical field, the current measured between the electrodes increases. At the centre of the measuring area, the microorganism creates a local minimum in the measured current, and when exiting the measuring area, the current decreases to the original level.

In a different embodiment, the electrodes may be arranged along the longitudinal extension of the channel, again in the same plane, and an alternating electrical field extends along a portion of the channel is generated. When a microorganism enters the area between these electrodes, the current measured between the electrodes decreases. At the centre of the measuring area, the microorganisms creates a local maximum in the measured current, and when exiting the measuring area, the current increases to the original level.

Irrespective of the electrode arrangement, by evaluating the sensor signal, e.g. the current signal, and specifically by detecting the above patterns in the current signal in response to a microorganism passing the sensor, a counter can be implemented counting the number of times such patterns is noticed in the signal of the impedance sensor.

In a different embodiment, the entire channel may be covered by the electrical field of an impedance sensor such that the overall number of microorganisms in the channel provide for an overall additive dipole to the electrical field applied. By means of comparing the signal of the sensor with the signal of a sensor that measures a channel not populated with microorganisms, at least it can be detected if microorganisms are present in the channel.

The sensor can also be referred to as a capacitive sensor that is driven by an alternating voltage. All of the above embodiments apply in the same way.

Preferably, the electrodes of the above impedance or capacitive sensor are not in direct contact with the fluid, as long as the desired portion of the channel and the fluid therein are exposed to the electrical field.

However, in a different embodiment, the detector may include an optical sensor for optically detecting the presence and/or amount of microorganisms in the fluid.

Preferably, an evaluation circuit is provided for processing the signals supplied by the sensor. Such evaluation circuit may be an electronic circuit separate from the carrier to be introduced later, but more preferably is embodied as electronic circuit integrated into the same carrier that supports the channel. The evaluation circuit may be considered as part of the counter if any given that it identifies counts in the signal supplied by the sensor of the counter. Or, the evaluation circuit may be considered as part of the valve if any to be introduced below given that an output signal of the evaluation circuit controls the valve.

The valve preferably is configured to close the inlet of the channel in order to prevent additional fluid sample to enter the channel, and hence additional microorganisms to enter the channel. The valve can basically be any valve, including a mechanical valve, a hydraulic valve, or a pneumatic valve. Preferably, the valve, irrespective of its above class, is electrically activated. Preferably, the valve is a solenoid valve. Preferably, a plug of the valve can be transferred from a sealed position where the inlet is sealed by the plug or an element the plug acts on, to an open position where the inlet is open to receive fluid, and vice versa. Preferably, such element can be a flexible layer e.g. in form of a foil or a membrane that is arranged between the plug and the inlet such that in response to activating the plug from the open position to the sealed position the plug is moved towards the inlet and thereby presses the flexible layer towards, and possibly to a certain extent into the inlet. Here, the plug is responsible for exerting a force on the flexible layer to seal the inlet. The plug preferably may take the form of a pin or a ball, a spherical end of which plug supports self-aligning of the plug.

Preferably, the device comprises a holder for the plug. Hence, the plug may be supported by the holder, which preferably is also true for an actuator for the plug. In one embodiment, the holder is an element separate from the carrier to be introduced below, and the holder and the carrier provide for an interface to be mechanically connected to each other, preferably in a releasable way, and preferably by maintaining a distance there between. Preferably, such mechanical interface is a snap-fit. In such a scenario, it is preferred that the holder including the valve and the actuator is a multi-use member, to which the carrier-structure combination can be attached as a single-use, and hence a disposable member. In a different embodiment, the mechanical valve is implemented on-chip, i.e. on the carrier, my MEMS structures.

The valve preferably is activated from its open position into its sealed position in response to the counter supplying as a result that a certain number of microorganisms are detected to have entered the channel. For this purpose, the evaluation circuit may determine when a threshold is met by comparing the counted number of microorganisms with the threshold. In response to reaching or exceeding the threshold, the evaluation circuit may electrically trigger the actuator of the valve to transfer the plug from its open position into its closed position. The threshold preferably is defined up-front and is considered as number of microorganism sufficient to apply further treatment on.

The device preferably comprises a carrier and a structure arranged on top of the carrier for at least co-defining the channel, and preferably for defining at least some walls of the channel.

The carrier preferably comprises a substrate, preferably a semiconductor substrate, and most preferably a silicon substrate, and a stack of conducting and non-conducting layers arranged on the substrate. The stack of layers may represent a CMOS stack of layers used in CMOS processing for contacting electronics integrated into the semiconductor substrate. Such layers include metal layers and/or insulating layers and/or passivation layers, the latter being made from silicon nitride or silicon dioxide. As indicated above, the carrier preferably may contain electronic circuitry integrated into the carrier, which circuitry may specifically comprise the evaluation circuit.

Accordingly, the structure contributing to the channel preferably is arranged on top of the stack of layers. In one embodiment, the structure is formed from a film coated onto the carrier, and specifically onto the stack of layers, which film is structured accordingly. E.g. a groove is formed in the film that co-defines the channel. In case such groove is manufactured, e.g. etched to the bottom of the film, the carrier, and specifically a top layer of the stack may be exposed and hence contribute as bottom wall of the channel. In case the carrier is prepared to implement electronic and/or electrical elements such as parts of the counter, a sensor for sensing properties of the microorganisms resident in the channel, a heater, etc., such elements may as needed be brought close to or even direct contact to the fluid in the channel which is enabled by the present configuration.

In addition to the film, a cover may be provided on top of the film and cover the groove, and hence defining a ceiling of the channel. In one embodiment, it may be preferred that the cover comprises two openings therein serving as inlet and as outlet of the channel. In a different embodiment, the inlet and the outlet of the channel are on the same level as the channel, i.e. are defined in the film.

According to the above embodiments, the channel, or parts of, may be made from organic polymers or from inorganic material such as SiO, SiN, or metal.

Given that the carrier preferably contains electrically conducting paths, such as provided in the stack of layers, it is preferred that electrical elements supporting the function of the device are made from the electrically conducting layers of the stack. For example, in case of an impedance or capacitive sensor, electrodes, and in particular two electrodes supplied with alternating voltage are arranged in the stack of layers, and preferably are made from a metal layer of the stack. The electrodes preferably are arranged such that the electrical field generated by applying a voltage to the electrodes penetrates the desired portions of the channel.

In a very preferred embodiment, the electrodes of the sensor are electrically connected, e.g. by conducting paths in the stack, to the circuitry preferably integrated in the carrier and representing the evaluation circuit.

In a preferred embodiment, the device comprises a sensor tor detecting a metabolic activity of the microorganisms in the channel. Owed to the small volume of microorganisms, a change of color or obfuscation invoked by such metabolic activity may not be reasonably detected in the present device. Hence, it is preferred to apply a sensor for detecting a pH scale of the fluid in the channel, i.e. the power of hydrogen ions in the fluid that stem from the metabolic activity of the microorganisms. Preferably, the sensor for doing so includes an ISFET (Ion Sensitive Field Effect Transistor), preferably integrated into the carrier. Such ISFET comprises source and drain, and a channel there between implemented in the semiconductor substrate of the carrier, as well as an ion sensitive gate electrode provided in the stack of layers. The gate electrode preferably reaches vertically through the stack of layers and is directly exposed to the fluid in the channel. In another embodiment, the sensor for detecting metabolic activity is an impedimetric sensor, such as a capacitive sensor, possibly including the evaluation of phase information.

Metabolizing microorganisms produce carbon dioxide which in the aqueous liquid in the channel such as the fluid sample or an added culture fluid is converted into bicarbonate and hydrogen ions (H⁺). The increase in the hydrogen ion concentration causes a decrease of the pH value of the liquid in the channel. In case of an ISFET, the hydrogen ions H⁺ act as an electric charge onto the gate of the ISFET which charge impacts the current through the channel between source and drain. By preferably detecting a change in the current of the ISFET a change in the pH value in the channel is detected, and as a result the metabolic activity of the microorganisms trapped in the channel is proven by the sensor.

Accordingly, an increase in the H⁺ concentration corresponding to a decrease of the pH of the medium in the channel is a quantitative measure of the metabolic activity of the microorganisms present in the channel. Therefore, the amount of protons, i.e. the extent of the decrease of the pH is dependent on the number of microorganisms and on how actively the microorganisms which are present metabolize. Advantageously, this may be exploited to test the effect of various conditions, including in particular in the presence or absence of antimicrobial drugs or in the presence of various concentrations of a particular antimicrobial drug or in the presence of variable medium such as variable carbon sources which may have a diagnostic value.

For example, addition of an effective antimicrobial drug to the liquid in the channel causes decrease in the metabolic activity of the microbes which is smaller compared to a medium without the antimicrobial drug or even results in no decrease in pH when microbial metabolism is totally inhibited because the microbes were killed. Antimicrobial drugs may include one of a bacteriostatic, a fungistatic, a bactericide, a fungicide. The first ones prevents the microbes from cleavage while the latter ones kills the microbes. These kind of drugs keep the pH value constant or make the pH value increase relative the ph value prior to adding the drug since in case of effectiveness the drug the metabolic activity at least does not increase.

For this reason, in some preferred embodiments the pH value of the fluid in the channel is measured continuously or at certain time points by the sensor including time intervals before and after having added an antimicrobial drug. In these and further preferred embodiments parallel measurements with variable conditions in the liquid of the channels and the same number of microorganisms collected from the fluid sample are performed.

Alternatively, or in addition to the pH sensor, one or more other sensors may be provided and arranged to sense one or more of an ion concentration other than hydrogen in the fluid in the channel, a glucose concentration in the fluid in the channel, or other biomolecules such as one or more of iron containing proteins, a lactate concentration, CO2, ethanol, antimicrobial agents, antibiotics, viruses, exosomes and fragments thereof, microorganisms, or cell walls, or fragments thereof, proteins, or antibodies, or enzymes, free nucleic acids, or fragments thereof, smaller molecules, acids, or bases, or salts, or sugars, for example, that may be of metabolic importance and are suited to prove metabolic activity.

In one embodiment, instead or in addition to the pH value, temperature of the fluid in the channel is measured given that subject to metabolic activity, temperature of the fluid in the channel may change.

In one embodiment, a gas sensor may be provided at the outlet of the channel, in addition to the pH sensor or alternative thereto. Such gas sensor may measure gases such as CO₂, or VOC emitted through the size selective filter to monitor metabolism of the microorganisms. The gas sensor may be embodied as electrochemical cell, or as a chemiresistor, e.g. containing gas sensitive metal oxide material.

In another embodiment, in addition or alternative to the ph sensor, one or more of the following sensors may be provided, preferably arranged on or in the substrate:
a photodetector for detecting optical parameters of the fluid in the channel which may change dependent on the metabolic activity of the microorganisms in the fluid in the channel; preferably, a frequency selective optical sensor may be provided that in one embodiment detects radiation at selected frequencies: such sensor may be employed in particular in case radiating, and specifically fluorescent labels are attached to microorganisms in the fluid sample, which may get there during preprocessing the fluid sample;
a humidity sensor, e.g. for sensing humidity in a space subsequent to the outlet;
a flow sensor for determining a flow of the fluid in the channel, and in particular for detecting a clogged or otherwise blocked channel—either by the microorganisms themselves when packed in the channel or by other substances—which may prevent addition of reagents; including, for example, a differential temperature sensor.

In particular, a flow sensor may be provided in combination with an impedance or capacitive sensor, which in one embodiment is the impedance or capacitive sensor or the detector. These sensor provide as results a mass flow on the one hand, and the number of microorganisms in the channel.

In one embodiment, a heater is arranged on or in the carrier, or is integrated into the carrier. Again, it is preferred that the carrier preferably contains electrically conducting paths, such as provided in the stack of layers. In case the heater is a resistive heater, the heater may be arranged in the stack of layers, and preferably be made from a metal layer of the stack. The heater preferably is arranged such to be thermally coupled to the channel. The heater preferably is connected, e.g. via conducting paths in the stack, to the integrated circuitry representing the evaluation circuit, which may control the operation of the heater. The heater may preferably be activated after concentrating and/or collecting the microorganisms in the channel, preferably for triggering and/or maintaining the metabolic process of the microorganisms.

In a preferred embodiment, another size selective filter is arranged at the inlet. This filter is designed to prevent particles much bigger than the microorganisms desired to be collected in the channel from entering the channel or from clogging the inlet. For this purpose, the other size selective filter comprises pores of a size bigger than an average size of the microorganisms to be counted by the counter. It turned out that for a majority of microorganisms to be detected, pores of the other size selective filter are preferably dimensioned with a diameter in the range between 1 μm and 100 μm to match this purpose. In one embodiment, the other size selective filter is attached to the carrier or structure. In a different embodiment, the other size filter is attached to the holder. In a further variant, the other size filter is attached to the injector, which in one embodiment may also be considered as element of the device, or the apparatus respectively. The filter may also include a filter cascade, or a two- or multi-level filter. In case that large volumes of liquid are processed by the device, preferably a two-step filtration is employed using a first filter with a large area collecting the wanted microorganisms. A smaller amount of liquid including the collected microorganisms is then transferred from there to the channel in a second step, at the end of which channel a second filter is applied. Both filters preferably are size selective filters as described above with the properties of retaining microorganisms. By means of such two-step filtration, the processing time is accelerated in view of only a small volume of liquid containing the microorganisms is supplied to the actual channel with the filter. The large volume of fluid does not need to be processed in combination with the small-scale filter at the end of the channel.

The carrier-structure combination may be considered as a chip, in particular when the carrier comprises a semiconductor substrate and circuitry is integrated in the carrier. Such chip preferably is of a dimension of 2 mm or less in length, of 0.5 mm or less in width, and of 0.5 mm or less in height including both the carrier and the structure. In another embodiment, the chip additionally comprises an encapsulation partly encapsulating the chip. Such encapsulation may be manufactured in a molding process. The encapsulation at least uncovers the inlet and the outlet of the channel in the structure. This may be achieved by corresponding protrusions in a mold applied during molding. This feature effects that the plug of the valve does not necessarily directly act on the inlet of the structure, but on the encapsulation instead which may be more robust to mechanical impact such that in particular in this case a flexible layer may not be needed between the inlet and the plug for damping purposes.

In one embodiment, a movement of microorganisms to the channel may be biased by an electric field arranged between the inlet or pre-inlet and the outlet or post-outlet in order to influence cell sorting and compensating non-symmetries of filter structure. A controlled voltage needed may be generated, on each chip as will be explained later on.

In another embodiment, microorganisms may be sorted prior to entering the channel, and preferably sorted based on dielectrophoresis.

The process of concentrating microorganisms in the channel may be considered sufficient for the processing of microorganisms as specified in the preambles of the claims even without further processing steps. Volumes are preferably so small that the metabolic activity of even one single—e.g. E. Coli—bacterium is enough to increase the H+ concentration in the channel, and in particular in the fluid in the channel containing the microorganism that the corresponding decrease in pH is detectable. For example, a pH value around a neutral pH, i.e. pH 7, of the fluid in the channel such as the original (body) fluid of the sample or of a generic culture fluid added to the channel may exhibit an decreased pH value by −0.3 within approximately 5 h. Note that a resolution of pH 0.05 is highly realistic to be detected by a pH sensor as is suggested later on.

According to a further aspect of the present invention, a method is provided for processing microorganisms. The method includes the steps of introducing a fluid sample into a channel. The channel is dimensioned to hold, between an inlet and an outlet, a volume in a range between 1 nl and 50 μl of fluid. Microorganisms present in the fluid sample are retained in the channel by means of a size selective filter arranged at the outlet of the channel. The size selective filter comprises pores of a size smaller than an average size of the microorganisms to be processed.

In a preferred embodiment, microorganisms present in the fluid sample introduced into the channel are automatically counted by means of a counter. Preferably, an electrically controllable valve is automatically activated thereby closing an inlet of the channel in response to a counting result provided by the counter.

In preferred embodiments, one or mere of the following steps are performed after the concentration of microorganisms is achieved in the channel, i.e. preferably after having closed the inlet of the channel: Supplying a generic culture fluid into the channel heating the fluid in the channel; adding a reagent into the channel; measuring a metabolic activity of the fluid in the channel by means of a sensor.

In the embodiment of adding a generic culture fluid—such as LB-broth or another broth or culture medium as known in the art—into the chamber filled by microorganisms it is aimed at triggering or maintaining metabolic activity of the microorganisms trapped in the channel. By adding such generic culture fluid, cells of the microorganisms may perform life sustaining chemical transformations and are kept alive for enabling later measurements involving the microorganisms. The generic culture fluid in one embodiment may be supplied via the inlet of the channel. Hence, after closing the inlet in response to the counter result, the injector may be removed from the inlet or a different part via which the fluid sample was transferred to the inlet. In a next step, another injector, again a syringe or a pump, for example, now filled with the generic culture fluid, may be applied to the subject interface after or preferably prior to reopening the valve, and preferably after allowing excess body fluid if any to be removed from the inlet. Excess fluid of the generic culture fluid may exit the channel via the outlet, and if needed may be collected at some basin or be blotted at an edge of the device near the outlet. Such excess generic culture fluid instead may also or in addition be one or more of blown off, drained and centrifuged/spun off at the outlet. The same removal operations may also be applied for removing excess body fluid sample after passing the outlet. The size selective filter at that stage may also be shut off with an additional cover in order to avoid drying out the channel, given that a certain amount of fluid is desired to keep the microorganisms alive in the channel. The culture fluid may in another variant be drawn into the channel via its inlet. This may be supported by a buffer material being inserted into the channel prior via its outlet. Thereafter, the inlet of the channel may be immersed into the culture fluid.

The generic culture fluid may in a different embodiment be supplied via the outlet, or, in a third variant may be pre-stored, either in some storage in the channel, or e.g. or in form of a saturated pad that may be applied to the filter at the outlet. By means of squeezing the pad, the culture fluid may enter the channel through the outlet.

Still, owed to the size selective filter at the outlet, the microorganisms are prevented from leaving the channel through the outlet.

In another variant, and preferably in addition to the supply of the generic culture fluid into the channel, the fluid in the channel preferably is heated. Such heating may evoke an incubation of the microorganisms in the channel. The heater previously introduced may be used for such heating step to heat, if required, to temperatures specific for the microorganism to be tested. Temperature control may also be adapted in response to a metabolic process of the microorganisms introduced by the supply of the generic culture fluid. Hence, subject to the testing activities envisaged, the fluid in the channel may be heated at any time desired.

In another embodiment, and preferably in addition to the supply of generic culture fluid and, if needed, the heating, and preferably after the supply of the generic culture fluid a reagent, and more preferably one or more of a growth reagent or an antimicrobial drug, such as a bacteriostatic, a bactericide, fungistatic or a fungicide is supplied into the channel. These reagents may be generic reagent or already selected specific to the microorganism which may even be identified upfront. Generally, other reagents that may in addition or instead be supplied into the channel include one or more of:
lysis buffer;
PCR primer;
DMA polymerase;
Desoxy-necleotides;
Luminescent labels;
Au nanoparticles with affinity molecules, such as antibodies;
affinity molecules, such as antibodies, labelled with luminescent labels;
acids and/or bases: For example, the pH value of fluid may be desired to be changed in case microorganisms still show metabolic activity after a descrease in the pH value which may indicate the presence of specific bacteria.

The reagent may in one embodiment be added to the channel via the outlet. In this way, it is taken advantage of the small size of reagents, which at least are smaller than the microorganisms, such that the reagents can pass the size selective filter at the outlet. In case the outlet and or a part connected to the cutlet provides an interface for an injector such as a syringe or a pump, the reagents may be supplied to the channel by attaching the injector to the outlet or the ether part and may be injected into the channel.

In a different embodiment, the device may be dipped into a liquid reagent such as liquid reagents with its outlet.

In another variant, the reagent may also be added by dispensing or ink-jet dropping into the channel. This may be achieved via the inlet of the channel in a non-closed state, or by an additional opening to the channel, that may also be closable.

In another variant, the reagent may be prepared in gel or dry form. It may be brought into contact with size selective filter membrane at the outlet of the channel and poured into the channel.

In case the reagent is prepared in gel or dry form, it may also be pre-deposited in the device, either in a deposit that can be opened, or directly in the channel, preferably in a quantity sufficient to survive intermediate washing steps.

The addition of reagents may include a stepwise increase of the reagent dosage into the channel, also referred to as titration. This helps in determining the dosage for treating a patient suffering from the infection, for example. Measurements of the metabolic activity may be taken at suitable points in time after each increase of the dosage, for example.

In the processing of the microorganism, it is preferred to detect a response to an antimicrobial drug applied (to the microorganisms) by measuring the drugs effect or a metabolic activity of the microorganisms. Such metabolic activity of the microorganisms in the channel can be detected by means of a sensor, and preferably the pH sensor, and more specifically the ISFET sensor introduced above.

In one specific example, the body fluid to be investigated is urine, a sample of which is collected from a patient, and filled into a syringe. In a preferred next step, the syringe is connected to the other size selective filter introduced above having a pore size larger, and preferably up to 20 percent larger than the largest microorganism to be tested, which may typically be in the order of 5 um-10 um. This assembly is connected to the device, and specifically to the interface of the device or the apparatus respectively. The urine is injected from the syringe into the channel or channels respectively in case of an apparatus containing multiple devices, until all volumes contain a predetermined amount of microorganisms—e.g. 10—and all inlets are shut. Next, the syringe is disconnected from the interface and unwanted excess fluid sample at the inlet/s is drained, e.g. by way of a suitable drainage. Then, the valve/s are opened again, and generic culture fluid (‘broth’) is added to all channels by means of another syringe. Excess generic culture fluid may be blotted from the outlets of the apparatus. The microorganisms are expected to maintain/begin with a metabolic activity (in which they undergo chemical processes) grow and optionally also proliferate and are considered to be alive. Such a quantitative measurement of metabolic activity of the microorganisms in the original (body) fluid may provide information about the number, state and their susceptibility to antimicrobial drugs as described below. In cases, where the original sample is a body fluid of a patient such quantitative measurement of a metabolic activity relates to the patient's medical condition and options for his treatment.

In a next step, an individual antimicrobial drug and/or growth reagent may be added to the channel by dipping an edge/area of the apparatus connected to the outlets into specific solutions such that these solutions may enter the channels via their outlets. Excess fluid may again be blotted from such edge.

In another step, the apparatus may be inserted into a chamber that makes the size selective filter be exposed to a controlled high humidity atmosphere such that a drying out of the small culture volumes is avoided. If needed, gases such as N2, CO2, or O2 are controlled in the atmosphere of the chamber. In a next step, or in combination with the previous step, volumes may be incubated at specific temperatures, e.g. at 37° C. During this procedure, the device/apparatus may be agitated, e.g. rotated e.g. to keep microbes suspended and aerate the culture liquid in the channel. During the processing described above the pH value in the channel may be measured over time, and specifically a gradual change of the pH may be detected over time and be evaluated. When applying an apparatus with multiple devices and applying different antimicrobial drugs to the different devices, finally channels are identified with viable microorganisms and channels are identified with non-viable microorganisms from pH evolution over time. The channels comprising with metabolically inactive, even non-viable microorganisms may be considered as susceptible to the antimicrobial drugs applied there which suggests a promising treatment for a patient suffering from an infection by them.

In another approach of processing of the microorganism, it is preferred to grew the microorganisms e.g. by adding a growth reagent. Again, preferably after having performed a first measurement of the metabolic activity of the microorganisms prior to adding the growth reagent, a response to the growth reagent can be detected by means of a second measurement after having added the growth reagent. The measurement results may be compared, and in case the second measurement results indicates an enhanced metabolic activity than the first measurement results, effectiveness of the growth reagent is proved.

In another embodiment, the channels may also be isolated in a ‘liquid sense’ by adding controlled volume of air at end of loading of volume with liquid such that the volume in the channel captured by the fluid is smaller than the channel volume

According to another aspect of the present invention, an apparatus is provided for processing microorganisms. The apparatus comprises multiple devices according to any of the preceding embodiments. These devices are arranged on or in a common platform. The platform preferably includes a reservoir which may serve as a common volume in fluid connection with each of the inlets of the devices. Hence, in case this reservoir is filled by the fluid sample, the fluid sample may enter the inlet of each device. Hence, instead of the inlets of the individual devices requiring an interface for the injector for the fluid, it is the platform that provides for an interface for the injector, preferably a syringe or a pump, which now is the interface common to all devices.

In preferred embodiment, a single filter pad represents the size selective filter for all devices.

According to another embodiment, each device comprises a counter configured to count microorganisms present in the fluid sample introduced into the channel, and an electrically controllable valve configured to close the inlet in response to a counting result provided by the counter, the valve comprising a plug.

Preferably, the apparatus comprises a common holder for the various plugs of the valves. Hence, in case a valve is implemented in form of a movable plug, each plug may be supported by the holder, which preferably is also true for the actuators for the plugs. It is preferred that each plug is individually controllable and hence is assigned an individual actuator. However, in a different embodiment, a single actuator may be arranged to control all the plugs in common. Hence, all inlets may show the same state at the same point in time, closed or sealed. Here, the different plugs may also be replaced by a common plug for closing all inlets at the same time. Such common plug may also take the shape of a ring or a block subject to the geometrical arrangement of the devices on the platform.

Preferably, the holder covers at least a part of the reservoir in the platform. In case the reservoir is built by a rim protruding from a plane of the platform, the holder may sit on the rim and co-define the reservoir built from the platform and the holder.

Again, in a preferred embodiment in between the plug and the inlet a flexible layer is arranged. In the apparatus embodiment, it is preferred that the flexible layer is arranged between the holder and the platform. Specifically, the flexible layer may be arranged in between the rim of the platform if any, and the holder, and preferably may be attached to one or both of the rim and the holder. The flexible layer preferably is not to cover any of the inlets in a relaxed state but is arranged distant from the inlets in the relaxed state, and hence enables the reservoir to be filled with fluid to be dispensed into the various inlets. Preferably, the flexible layer is a flexible layer common to all the valves of the apparatus, i.e. is not separated but a single piece. The flexible layer preferably is tight to any liquids given that the purpose of the flexible layer is to prevent liquid, and preferably also gas, from entering the channel if a desired number of microorganisms is detected to be trapped in the channel.

In one embodiment, the flexible layer is applied, e.g. laminated onto the holder. In different embodiment, the flexible layer is attached to the rim of the platform.

In one embodiment, the holder is made from injection molding, and hence is a mold compound. The holder may comprise a mechanical coding allowing the holder to connect to the platform only in a defined position and/or orientation. In one embodiment, the valve per device may comprise a ball and a pin. The pins may be supported in corresponding holes reaching through the holder. A ball each may be set onto each hole of the holder of a common plane. The balls each have a diameter exceeding the diameter of the hole such that, the balls do not fall into the holes but sit there on. In an embodiment, the holes may expand at their ends such that the balls partly or fully are absorbed by the holes at the respective ends. After having applied the balls to the holders, the flexible layer may be attached to the holder above the balls.

In a preferred embodiment, the platform not only mechanically supports the various devices but also serves for electrically connecting the devices, either amongst each other or to the environment. For this purpose, it is preferred that the platform comprises a lead-frame the individual devices are attached to, e.g. on die pads of such lead-frame. The devices, which in this respect may be considered as chips or dies may then be electrically connected to leads of the lead-frame, e.g. by wire bonding. For this purpose, each device may have contact pads exposed from the carrier. For example, areas of the metal layers of the stack may be exposed which in turn means that above the contact pads the film is removed in order to allow access to the contact pads. Such contact pads may then be provided with bond wires that reach down to the leads of the lead-frame. The platform itself may have contact pads, e.g. for contacting to the outside world, e.g. a computing system, a recording system, or both, for further processing or storing the results of the testing. In case of a lead-frame, the lead-frame preferably is encapsulated, at least where it makes sense.

Hence, the platform in combination with the chips may in one embodiment form one of the following packages, without limitation: LGA (Land Grid Array), COB (Chip On Board), QFN (Quad Flat No Leads). Also in case in individual devices, such device may be packaged in one of the above mentioned packages. As introduced for the apparatus, an individual carrier-structure combination may be attached to a die pad of a lead-frame, and electrically connected to leads of the lead-frame prior to being partially encapsulated by a mold compound, for example.

Hence, in one embodiment, at least the inlets of the various channels facing the recess are accessible, and hence are exposed from the platform, as well as the outlets. In a very preferred embodiment, the devices are assumed to have longitudinal axis coinciding with the longitudinal axis of the channel. Each device may be arranged in the platform such that the rim defining the recess crosses the device orthogonal to its longitudinal axis. This in turn guarantees that fluid injected or present in the reservoir does not reach into the outlets of the devices which otherwise may contaminate the microorganisms collected in the individual channels. This is even more true in case the holder for the plugs sits on the rim and seals the volume defined by the recess versus the exterior, and hence versus all the cutlets of the devices residing outside the rim.

Specifically in such embodiment, but also in other variants of the apparatus, it is preferred that the size-selective filter per device is embodied as a size-selective filter membrane common to all devices arranged in the platform.

Preferably, the cutlets of the various devices, covered by the common filter membrane, lead into a collection area, either individual per device, or common for all devices, that allows a temporary collection of fluid exiting the channel through the outlets and the filter. The collection area may in one embodiment be defined as a recess in the platform that allows to hold a certain amount of fluid therein, or leads into a drainage for draining such fluid into an external vessel. And/or the collection area may be equipped with an absorptive tissue which can be removed and replaced. In a different embodiment, the collected fluid can be actively evacuated or blown away. This removal of fluid can be performed automatically, in one embodiment.

By means of the embodiments of the apparatus introduced above, several volumes/channels may be implemented on a single apparatus instead of using several individual devices separate from each other. In this case, in one favorable implementation the fluid sample may be transferred from a syringe through a hole in platform serving as interface to the apparatus, and more specifically to the inlets of the various devices.

The devices, which in one example may be a set of devices in the range between 2 and 10, may be arranged in one or more linear rows in the platform, e.g. forming a square, or may be arranged circular. In the latter case, the recess preferably has a circular shape, too, and the filter membrane if any is of an annular shape.

Owed to the set-up of the apparatus, the same body fluid sample is injected into all channels of the devices integrated in the apparatus. Hence, the concept of the apparatus includes simultaneous multiple measurements of the same sample for enhancing reliability of the testing. In case the testing processes per device are finalized for each device, which in one embodiment may result in a statement that x devices detect microorganisms in the body fluid sample while y devices do not, with N being the overall number of devices, and y=N−x, a decision, such as a majority decision, may lead to a final result if metabolic microorganisms are detected as present in the body fluid sample or not. The same holds for the effectiveness of antimicrobial drugs applied to the collected microorganisms in all devices. In case the same antimicrobial drug is applied to the channels of all the devices, e.g. a majority or different decision can be taken if this antimicrobial drugs is considered as effective in treating the subject microorganisms collected in the channels. In a different variant, different antimicrobial drugs are applied to if devices, and hence, to different channels of the apparatus. E.g. a different antimicrobial drugs may be applied to each channel. In this approach, the effectiveness of different antimicrobial drugs can be evaluated, and in particular it can be detected which of the antimicrobial drugs applied are effective and which are not.

In the above embodiments, a growth phase as required in conventional processing systems no longer is required. This saves a significant amount of the entire processing time.

The presence of microorganisms in body fluid samples can be detected at the point of care. No large instruments are required. The device and/or the apparatus is portable, and can be used not only in the hospital but also close to the patient, at the point of care.

### BRIEF DESCRIPTION OF THE DRAWINGS

Embodiments of the invention will be better understood and objects other than those set forth above will become apparent when consideration is given to the following detailed description thereof. Such description makes reference to the annexed drawings, wherein:

FIG. 1 shows a longitudinal out view of a device according to an embodiment of the present invention,

FIG. 2 shows a lateral cut view of a the device of FIG. 1 along line B-B′,

FIG. 3 illustrates a perspective view of an apparatus according to an embodiment of the present invention,

FIG. 4 shows an exploded perspective view of an apparatus according to another embodiment of the present invention,

FIG. 5 shows the exploded apparatus of FIG. 4 from a different perspective.

FIG. 6 shows a perspective view of an apparatus according to a further embodiment of the present invention,

FIG. 7 shows the platform 7 isolated from the apparatus of FIG. 6 in a perspective view, and

FIG. 8 illustrates a method for processing microorganisms according to an embodiment of the present invention.

### DETAILED DESCRIPTION OF THE DRAWINGS

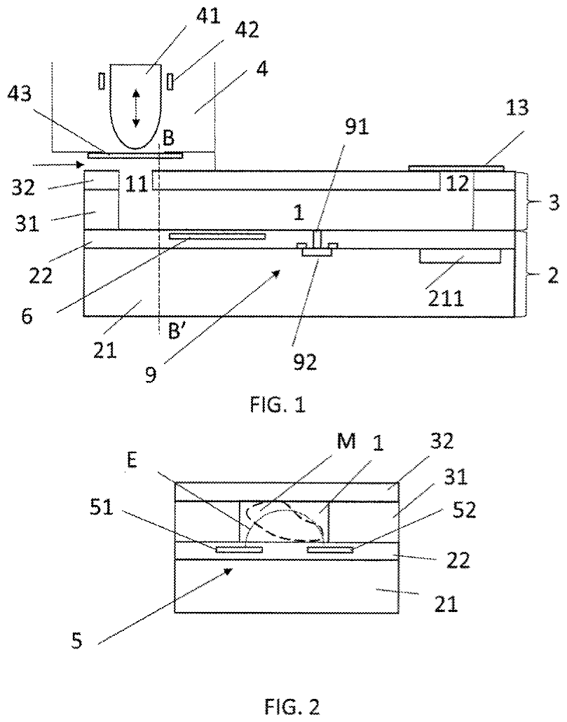

FIG. 1 illustrates a longitudinal cut view of a device according to an embodiment of the present invention, while FIG. 2 shows the according lateral cut view along line B-B′. The device comprises a carrier 2 including a substrate 21, and in particular a silicon substrate, and a stack 22 of layers deposited on top of the substrate 21, which stack 22 includes conducting electrically isolating and conducting layers of a CMOS layer stack to be used in CMOS processing. A structure 3 is arranged on top of the carrier 2. The structure 3 comprises a film 31, such as an SU8 film or equivalent, which is structured e.g. by means of etching. In particular, a groove is structured in the film 31 contributing to a channel 1. The structure 3 further comprises a cover 32, made from a dry film, for example, covering the film 31 including the channel 1 except for two openings and representing an inlet 11 and an outlet 12 of the channel 1.

The device further comprises a holder 4 including parts of a valve for controlling an influx of fluid sample into the channel 1. The fluid sample to be introduced into the channel is illustrated by an arrow close to the inlet 11. The holder 4 comprises a plug 41 movable in two directions as indicated by the double arrow. The plug 41—which can also be referred to as pin ion the present embodiment—is controlled ion its position and movement by an actuator 42, such as an electromagnetic actuator, or a piezoelectric actuator. An opening of the holder 4 is covered by a flexible membrane 43 that in particular is flexible enough to close the inlet 11 in response to the plug 43 pushing the flexible membrane 43 towards the inlet 11 thereby closing the inlet 11 and preventing further fluid to enter the channel 1 via the inlet 11. The holder may be attached to the structure 3—carrier 2—which presently only is schematically indicated by some line, but which attachment in practice will be a more robust and appropriate attachment, preferably releasable.

At the outlet 12 of the channel 1, a size selective filter 13 is arranged. In the stack 22 a heater 6 is provided in form of a resistive heater 6 controlled by an evaluation circuit 211 integrated into the substrate 21. A sensor 3 represents an ISFET integrated into the substrate 21 including source and drain and an ISFET channel 92 in between, and a gate electrode 91 controlling conductivity in the ISFET channel 92. The gate electrode 91 reaches through the stack 22 and is exposed to the fluid in the channel 1.

As can be derived from FIG. 2, in addition electrodes 51 and 52 are arranged in the stack 22 and are built from a metal layer of the stack 22. The electrodes 51 and 52 belong to an impedance sensor 5 that is arranged and configured as part of a counter: By means of the counter, microorganisms entering the channel 1 are counted by measuring their impedance. FIG. 2 shows an example of a microorganisms M present in the channel 1. By applying an AC voltage to the electrodes 51 and 52, electrical files lines such as schematically indicated by the dotted line E penetrate the channel 1 and hence the microorganism M in the channel 1. Since the microorganism M shows an impedance different to an impedance of the body fluid without any microorganism therein, the microorganism can be detected by means of the impedance sensor 5. A signal of the impedance sensor 5 may be evaluated in the evaluation circuit as to changes of the impedance in the signal. Any significant pair of changes may be interpreted as a microorganism passing the subject location of the impedance sensor 5.

In operation, body fluid sample is injected into the inlet 11 of the channel 1 and passes the impedance sensor 5, where microorganisms M present in the fluid sample passing are counted. The microorganisms M move on until being stepped by the size selective filter 13. The medium, i.e. the fluid sample filtered from the microorganisms M passes the filter 13 and exits via the outlet 12. In response to a sufficient amount of microorganisms counted, the evaluation circuit triggers the actuator 42 of the valve to move the plug 41 against the inlet 11, thereby closing the channel 1 for receiving further fluid sample. In further steps, culture fluid may be added to the channel, e.g. via the outlet 12, and the heater may be turned on for heating the fluid in the channel 1 and causing a metabolic activity of the micro-organisms M in the channel 1 which is measured by the sensor 9 in an pH measurement. At some point in time, an antimicrobial drugs may be added, e.g. via the outlet 12 again, and after some time the pH value of the fluid/microorganism in the channel 1 is measured again to find out if the applied antimicrobial drugs has lowered or stopped the metabolic activities of the microorganisms M.

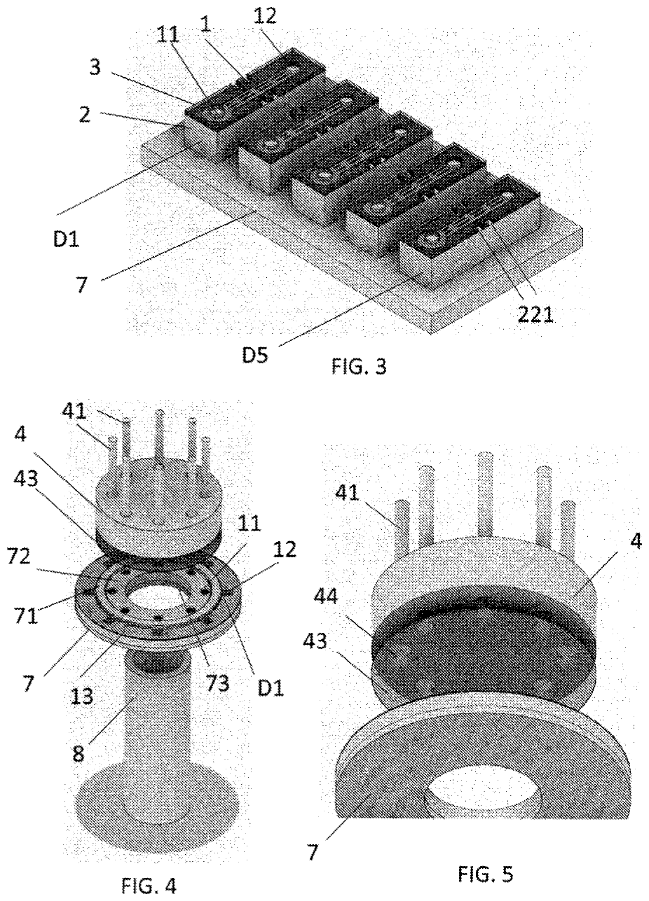

FIG. 3 illustrates a perspective view of an apparatus according to an embodiment of the present invention, comprising five devices D1 . . . D5, such as introduced in FIGS. 1 and 2, for example. Each device may be dimensioned of 2 mm in length and 0.5mm in width, and 0.5 mm in height. The devices D1 . . . D5 are arranged on a platform 7, such as an encapsulated lead-frame, or a printed circuit board. Each device comprises a carrier 2 and a structure 3 on top of the carrier 2 defining the channel 1 per device. The structure 3 comprises a film and a cover, the latter being depicted transparent, in order to allow in view into the channel 1. The film structuring the channel 1 also contains ridges protruding into the channel 1 for supporting the cover such that the channels do not collapse under hydrostatic pressure during a molding process that may be applied for building the channels.

In the present embodiment, contact pads 221 are exposed from the carrier 2, and in particular from the stack 22 of the carrier 2, by local removal of the structure 3. These contact pads 221 are wire bonded to the platform 7 (not shown) in order to electrically connect each device to the outside world. The present apparatus may in one embodiment be molded for forming an en capsulation, e.g. for protecting the wire bonds and the chips as such. However, the inlets 11 and the outlets 12 remain free from such encapsulation, if any.

Valves for controlling the filling of the individual channels 1 are not explicitly shown in FIG. 3, however, could be implemented by means of an external common holder for all plugs, or by micro-machined valves manufactured in the structure 3, for example.

FIG. 4 shows a perspective exploded view of an apparatus according to another embodiment of the present invention. The apparatus comprises a holder 4 and a platform 7. The platform 7 is of annular shape and supports eight devices Dx, as introduced in FIG. 1, for example. The devices Dx, only one of which is labeled as D1, are deposited on a lead-frame of the platform 7 (not shown), and are electrically connected thereto prior to the lead-frame being encapsulated by a mold compound giving the platform 7 the shape as presented. The eight devices Dx—which could be any number—are arranged radially each with the inlet 11 and the outlet 12 being separated by a circular rim 71. The rim 71 forms, at the side of the inlets 11 a reservoir 72, in particular in combination with a syringe 8 to be injected into an opening 73 in the platform 7. In the following, the syringe may inject a body fluid sample into the reservoir 72 from where the fluid sample enters the inlets 11 of the individual devices Dx. Outside the rim 72, the platform 7 provide for an annular area, also referred to as edge of the apparatus which is covered by an annular membrane serving as size selective filter for all outlets 13. This membrane prevents microorganisms from passing but allows excess fluid sample to pass, and be blotted from this outside area, for example. A holder 4 including eight pins 41 serving for valves for each inlet 11. The valves can be actuated by an actuator (not shown). As can be better derived from FIG. 5, the holder 4 comprises at its bottom side a sphere 44 for each opening holding one of the pins 41. The balls are dimensioned net to be swallowed by the openings but to rest in the openings. A flexible membrane 43 is provided and covers the spheres 44 made from PDMS or an equivalent sealing material. The holder preferably is injection molded, the openings for the pins are preferably of 0.5 mm diameter.

In a final assembly, the holder 4 is attached to the platform 7, e.g. by an adhesive. Preferably, the holder 4 and the flexible membrane 43 sit on the rim 71 of the platform. In operation, the pins 43 may be actuated by a spring/solenoid mechanism, and may be non-disposable. The filter 13 preferably has pores with a pore diameter in a range between 0.5 um and 1 um for holding back microorganisms. The platform 7 may have a diameter if approximately 10 mm.

Movement of microorganisms to the channels 1 may be biased by an electric field between a rotational axis of smart filter coinciding with the longitudinal axis of the syringe and an edge of the platform 7 in order to influence microorganism sorting and compensating non-symmetries of the apparatus structure. Controlled voltage needed may be generated on each device, while the rotational axis may be set to common voltage (e.g. GND).

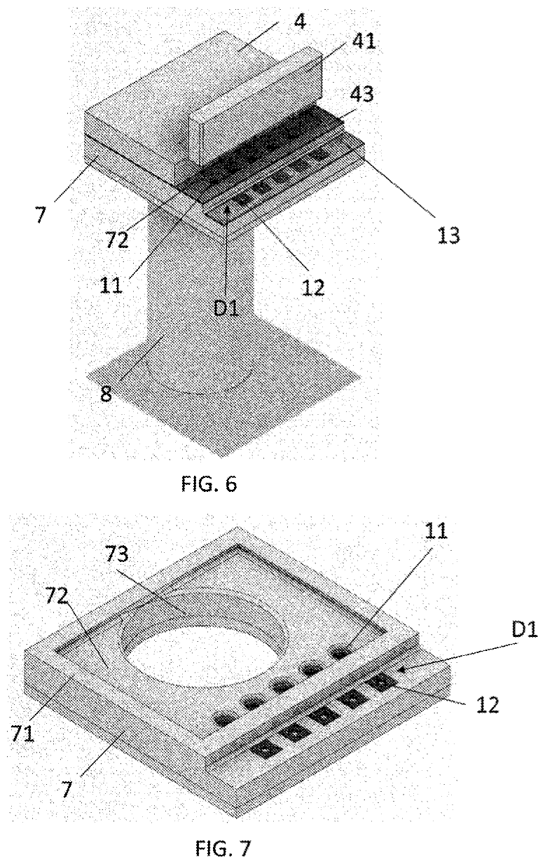

FIG. 6 shows a perspective view of an apparatus according to a further embodiment of the present invention, and FIG. 7 shews the platform 7 isolated from the apparatus of FIG. 6 in a perspective view.

In this apparatus, the platform 7 is of square shape, and the devices Dx are arranged linear in a row at one edge of the platform 7. Again, a rim 71 provides for a reservoir 72 in which reservoir an opening 73 is provided as interface for the syringe 8 to be attached and filling the reservoir 72 with a body fluid sample. The presently five devices are integrated in the platform 7 in a row with the inlets 11 being accessible from the reservoir 72, and the outlets 12 being arranged at the outside of the rim 72 in an area for collecting excess fluid. As can derived from FIG. 6, a filter 13 covers the outlets 12 for preventing microorganisms collected in the channels to leave. In FIG. 7, the filter no longer is shown to allow a view on the outlets 12. The filter 13 is a single piece filter 13 applicable to all outlets 12.

A holder 4 is attached to the platform 7 for supporting a plug 41 which presently is a plug 43 in form of a block common and applicable to all inlets 11 of the various devices. The holder 4 in addition may comprise an actuator (not shown) by which the block-like plug 41 may be lowered and re-lifted in order to close the inlets 11 and re-open all at one time.

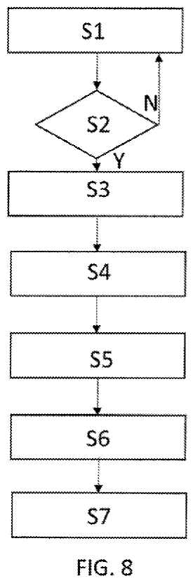

FIG. 8 illustrates a method for processing microorganisms according to an embodiment of the present invention, preferably by means of one of the devices or apparati according to any of the embodiments introduced above. Although the following embodiment is explained for a device, it is applicable to an apparatus, too, wherein all devices of the apparatus are filled by the same body fluid.

In step S1, a fluid sample is introduced into a channel of the device, and in response to any microorganisms present in the fluid sample, these microorganisms are counted in a counter indicating a present counting result. In step S2, it is verified if the counter result exceeds a threshold. If not (N), more fluid sample is introduced in the channel, and microorganisms are continued to be counted automatically. If at one point in time the counter result exceeds the threshold (Y), a valve is automatically triggered in step S3 to shut the inlet of the channel and prevents further fluid sample and hence further microorganisms from, entering the channel.

In step S4, after the inlet of the channel is closed by the valve, culture fluid is introduced into the channel for supporting a metabolic process of the one or more microorganisms trapped in the channel, e.g. via the outlet of the channel. Thereafter, e.g. at a defined interval after having added the culture fluid, in step S5 a first measurement is taken as to the fluid including microorganisms in the channel: It is measured if a metabolic process of the one or more microorganisms retained in the channel (1) takes place. This is performed e.g. by a sensor measuring the pH value in the channel.

After having conducted the first measurement, and preferably only if metabolic activity is measured in the first measurement, in step S6 antimicrobial drugs is supplied into the channel, preferably via its outlet.

Thereafter, e.g. at a defined interval after having added the antimicrobial drugs, in step S7 a second measurement is taken as to the fluid including microorganisms in the channel: it is measured again if a metabolic process of the one or more microorganisms retained in the channel still takes place. This is performed by the same sensor measuring the pH value in the channel.

In step S7, the results of the first and the second measurements are compared. In case the first measurement indicates metabolic activity while the second measurement does not, it can be assumed that the antimicrobial drugs stops metabolic activities of the microorganisms, and is suited as treatment for the patient. In case the first measurement indicates metabolic activity while the second measurement still does, it can be assumed that the antimicrobial drugs does not stop metabolic activities of the microorganisms, and as such is not suited as treatment for the patient.

1. A device for processing microorganisms, comprising
a channel comprising an inlet for introducing a fluid sample into the channel, and an outlet, wherein the channel is dimensioned to hold, between the inlet and the outlet, a volume in a range between 1 pl and 100 μl of fluid,
a size selective filter arranged at the outlet for retaining microorganisms in the channel,
wherein the size selective filter comprises pores of a size smaller than an average size of the microorganisms to be processed,
a sensor for detecting a metabolic process of the one or more microorganisms in the channel,
wherein the sensor is configured to detect one or more of the following in the fluid in the channel:
an ion concentration other than hydrogen,
microorganisms, or cell walls, or fragments thereof,
molecules smaller than 1000 Daltons, preferably including one of such as CO₂, ethanol, or VOC,
salts, or sugars.

2. A device for processing microorganisms, comprising:
a channel comprising an inlet for introducing a fluid sample into the channel, and an outlet wherein the channel is dimensioned to hold, between the inlet and the outlet, a volume in a range between 1 μl and 100 μl of fluid,
a size selective filter arranged at the outlet for retaining microorganisms in the channel,
wherein the size selective filter comprises pores of a size smaller than an average size of the microorganisms to be processed,
a detector for detecting microorganisms in the channel,
wherein the detector includes a sensor for detecting microorganisms in the channel,
wherein the detector includes a counter comprising the sensor, which counter is configured to count microorganisms (M) present in the fluid sample introduced into the channel.

3. The device of claim 2, comprising
an electrically controllable valve configured to close the inlet in response to a counting result provided by the counter.

4. The device of claim 1, comprising,
a carrier for the channel,
a structure arranged on top of the carrier at least co-defining the channel,
wherein the structure includes:
a film including a move reaching down to the stack, the groove representing the channel, and
a cover on top of the film covering the groove and comprising openings serving as inlet and outlet of the channel.

5-6. (canceled)

7. The device of claim 1, comprising:
a carrier for the channel,
a structure arranged on top of the carrier at least co-defining the channel,
wherein the carrier comprises a substrate and a stack of conducting and non-conducting lavers arranged on the substrate,
wherein the structure contributing to the channel is arranged on top of the stack of layers,
wherein the sensor is arranged on or in the carrier and comprises electrodes,
wherein the electrodes are arranged coplanar with respect to the channel, and are made from one or more of the conducting layers of the stack, or
wherein the electrodes are arranged at opposite walls of the channel.

8. (canceled)

9. The device of claim 1, wherein the size selective filter comprises pores with a diameter in a range between 0.01 μm and 10 μm.

10. The device of claim 1, comprising:
another size selective filter arranged at the inlet,
wherein the other size selective filter comprises pores of a size bigger than an average size of the microorganisms to be counted by the counter, and
wherein the other size selective filter comprises pores with a diameter in a range between 1 μm and 100 μm.

11. The device of claim 3,
wherein the valve includes a plug,
the device further comprising,
a flexible laver arranged between the inlet and the plug for closing the inlet in response to the plug pressing on the flexible layer.

12-14. (canceled)

15. The device of claim 1,
wherein the sensor is configured to detect a pH value of the fluid in the channel,
wherein the sensor comprises one or more of an impedance sensor, an electrochemical cell, or an ISFET,
wherein the ISFET is implemented in the semiconductor substrate and the stack,
wherein a gate electrode of the ISFET is exposed to the fluid in the channel.

16. The device of claim 1,
wherein the sensor comprises one or more of the following:
a gas sensor at the outlet of the channel,
a photodetector for detecting optical parameters of the fluid in the channel,
a humidity sensor,
a temperature sensor for measuring a temperature of the fluid in the channel.

17. An apparatus for processing microorganisms, comprising:
a platform including a reservoir,
multiple devices of claim 1 arranged on or in the common platform with the inlet of each device being in fluid communication with the recess,
wherein the platform comprises an interface configured to attach an injector for injecting a fluid sample into the reservoir.

18. The apparatus of claim 17, comprising:
a single filter pad representing the size selective filter for all devices.

19. The apparatus of claim 17, comprising per device:
a counter configured to count microorganisms present in the fluid sample introduced into the channel, and
an electrically controllable valve configured to close the inlet in response to a counting result provided by the counter, the valve comprising a plug,
wherein the apparatus comprises a holder for the multiple plugs,
wherein the holder is attached to the platform and holds an actuator assigned to each plug for evoking a movement of the subject plug.

20. The apparatus of claim 19, comprising:
a flexible layer arranged between the holder (4) and the platform,
wherein the plugs are arranged in the holder as to press the flexible layer against the assigned inlet in response to an activation of the corresponding actuator,
wherein the flexible layer is a single piece.

21. The apparatus of claim 17,
wherein the platform comprises a lead-frame,
wherein the devices are arranged on the lead-frame and are electrically connected to the lead-frame, and
wherein the lead-frame is at least partially encapsulated by an encapsulation.

22. Method for processing microorganisms, comprising:
introducing a fluid sample into a channel dimensioned to hold, between an inlet and an outlet a volume in a range between 1 nl and 50 μl of fluid,
retaining microorganisms in the channel by means of a size selective filter arranged at the outlet of the channel,
wherein the size selective fitter comprises pores of a size smaller than an average size of the microorganisms to be processed,
detecting a metabolic process of the one or more microorganisms in the channel by means of a sensor,
wherein the sensor is configured to detect one or more in the fluid in the channel:
an ion concentration other than hydrogen,
microorganisms, or cell walls, or fragments thereof,
molecules smaller than 1000 Daltons, preferably including one of CO₂, ethanol, or VOC,
salts, or sugars.

23. Method according to claim 22, comprising:
automatically counting microorganisms present in the fluid sample introduced into the channel by means of a counter,
automatically activating an electrically controllable valve thereby closing an inlet of the channel in response to a counting result provided by the counter.

24. Method according to claim 22, comprising:
after closing the inlet of the channel supplying culture fluid into the channel for supporting a metabolic process of the one or more microorganisms in the channel,
wherein the culture fluid is supplied to the channel via its outlet.

25. (canceled)

26. Method according to claim 22,
measuring the metabolic process of the one or more microorganisms retained in the channel by means of the sensor in a first measurement,
wherein the metabolic process of the one or more microorganisms retained in the channel is measured after the culture fluid is supplied to the channel,
supplying a reagent into the channel, and
measuring the metabolic process of the one or mote microorganisms in the channel by means of the sensor in a second measurement,
wherein the reagent is supplied into the channel via its outlet.

27-34. (canceled)
